(19) United States
(12) Patent Application Publication
(10) Pub. No.: US 2026/0235552 A1
(43) Pub. Date: Aug. 13, 2026

(54) METHODS AND SYSTEMS FOR DOPANT-ASSISTED SPECTROMETRY

(71) Applicant: Analytical Detection LLC, Lake Forest, CA (US)

(72) Inventors: Sean Michael Mathisen, Fountain Valley, CA (US); Naomi Jordan Hernandez, Whittier, CA (US); Andrey N. Vilkov, Aliso Viejo, CA (US)

(21) Appl. No.: 19/224,539

(22) Filed: May 30, 2025

(51) Int. Cl. G01N 27/622; G01N 27/623; G01N 33/94

(52) U.S. Cl. CPC G01N 27/622; G01N 27/623; G01N 33/946; G01N 33/9486

(57) ABSTRACT

A technique for detecting an analyte in a sample may include contacting the sample with at least one dopant. The sample includes the analyte. The at least one dopant includes a compound of Chemical Formula 1:

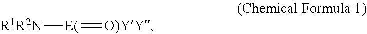

where E is independently phosphorus (P), sulfur(S), or arsenic (As), where Y′ and Y″ are each independently a chemical species or a lone pair of electrons, and where R¹ and R² are each independently selected from hydrogen, an alkyl group, an aryl group, a substituted alkyl group, or a substituted aryl group. The technique may further include forming an adduct ion from the analyte and the at least one dopant. The technique may further include performing spectrometric analysis of the adduct ion. The technique may further include determining, based on the spectrometric analysis, the analyte.

### TECHNICAL FIELD

The present disclosure generally relates to methods and systems for detecting analytes using dopants, for example, using spectrometry.

### BACKGROUND

Trace detection systems are designed to collect, analyze, and identify minute amounts, such as picograms or nanograms, of substances of interest that may be completely invisible to the unaided eye. Substances of interest to be detected by trace detection systems include explosives, drugs, chemical weapons, and toxic industrial chemicals. A sample including trace amounts of an analyte may be collected, and vaporized. The vaporized sample may be ionized and subsequently analyzed. Ion Mobility Spectrometry (IMS) and Mass Spectrometry (MS) trace detectors are commonly used methods to detect explosive, narcotics, and chemical weapon threats with high sensitivity and rapid analysis capabilities.

### SUMMARY

The present disclosure describes systems, techniques, and devices for detecting an analyte in a sample using a dopant configured to interact with the analyte.

Detection techniques for chemical substances may use agents such as dopants for detecting illicit substances, such as narcotics, pesticides, and chemical warfare agents, through spectrometric analysis. Dopants may be used to enhance the specificity of the technique. For example, a dopant may be added to a carrier gas to promote the ionization and detection of analyte molecules. Different dopants exhibit varying degrees of chemical selectivity towards specific analyte classes. For example, some dopants may preferentially ionize explosives, while others may be more effective in ionizing drugs. The choice of dopant can therefore be tailored to target specific types of analytes, enhancing the specificity of the spectrometric system for particular applications.

In some examples, an example method for detecting an analyte in a sample includes contacting the sample with at least one dopant. The sample includes the analyte. The at least one dopant includes a compound of Chemical Formula 1:

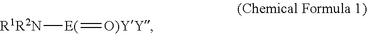

where E is independently phosphorus (P), sulfur(S), or arsenic (As), where Y′ and Y″ are each independently a chemical species or a lone pair of electrons, and where R¹ and R² are each independently selected from hydrogen, an alkyl group, an aryl group, a substituted alkyl group, or a substituted aryl group. The method may further include forming an adduct ion from the analyte (e.g., in the sample) and the at least one dopant. The method may further include performing spectrometric analysis of the adduct ion. The method may further include determining, based on the spectrometric analysis, the analyte.

In some examples, an example ion mobility spectrometry system includes a dopant chamber configured to fluidize a dopant composition to generate at least one dopant. The system may further include a drift chamber configured to receive an adduct ion comprising an ionized product of the at least one dopant and an analyte. A sample includes the analyte. The system may further include an ion detector configured to generate a signal in response to detecting drifted ions. The system may further include processing circuitry configured to determine, based on the signal, a sample drift time. The processing circuitry may be further configured to detect the analyte in the sample by comparing the sample drift time with a target drift time. The at least one dopant includes a compound of Chemical Formula 1:

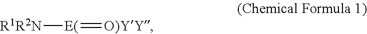

where E is independently phosphorus (P), sulfur(S), or arsenic (As), where Y′ and Y″ are each independently a chemical species or a lone pair of electrons, and where R¹ and R² are each independently selected from hydrogen, an alkyl group, an aryl group, a substituted alkyl group, or a substituted aryl group.

In some examples, an example sampling swab is pre-treated with at least one dopant agent. The at least one dopant may include a compound of Chemical Formula 1:

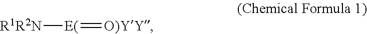

where E is independently phosphorus (P), sulfur(S), or arsenic (As), where Y′ and Y″ are each independently a chemical species or a lone pair of electrons, and where R¹ and R² are each independently selected from hydrogen, an alkyl group, an aryl group, a substituted alkyl group, or a substituted aryl group.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a block diagram illustrating an example system for ion mobility spectrometry, in accordance with one or more aspects of this disclosure.

FIG. 2 is a flow diagram illustrating an example method for ion mobility spectrometry, in accordance with one or more aspects of this disclosure.

FIG. 3 is a block diagram illustrating an example computing device configured to control a system for ion mobility spectrometry, in accordance with one or more aspects of this disclosure.

FIG. 4 is a plot illustrating example ion mobility plasmagrams of samples including fentanyl in presence and absence of a dopant, in accordance with one or more aspects of this disclosure.

FIG. 5 is a plot illustrating example ion mobility plasmagrams of samples including acrylfentanyl in presence and absence of a dopant, in accordance with one or more aspects of this disclosure.

FIG. 6 is a plot illustrating example ion mobility plasmagrams of samples including xylazine in presence and absence of a dopant, in accordance with one or more aspects of this disclosure.

FIG. 7 is a plot illustrating example ion mobility plasmagrams of samples including or excluding 4-anilino-N-phenethylpiperidine in presence and absence of certain dopants, in accordance with one or more aspects of this disclosure.

### DETAILED DESCRIPTION

The present disclosure describes techniques for dopant-assisted spectrometric analysis.

The specificity of a spectrometric technique or system refers to its ability to selectively detect and differentiate between different ions in a sample (e.g., an analyte or a mixture). Spectrometric analysis may be used for the detection and identification of trace amounts of chemicals, including drugs, explosives, and volatile organic compounds. The specificity of spectrometric techniques or systems arises from the combination of several factors, including chemical selectivity and measurement accuracy.

Adducts result from the combination of two or more distinct molecules (for example, an analyte and a dopant) into a single reaction product, substantially preserving the atomic composition of all constituent molecules and forming a unique molecular species. These molecular species may exist as positive or negative ions, with corresponding adduct ions formed in either positive or negative ion modes to enhance the sensitivity of spectrometric devices for specific classes of target compounds.

The use of adducts may facilitate ionization by enabling ion attachment to substances that are only weakly ionizable or entirely non-ionizable. Additionally, adduct formation stabilizes fragile molecular ions that might otherwise undergo fragmentation during analysis, reducing signal interference and improving detection sensitivity. Furthermore, the binding of a dopant molecule to the target substance shifts its spectral signal away from background chemical noise, thereby increasing the specificity and sensitivity of the detection technique. Dopant agents in adduct ion formation may be used for various applications, including in explosive trace detection. Certain dopants include chlorinated and ammoniated dopants. Organophosphorus compounds may also be used as dopants for detecting secondary and tertiary amines. However, such dopants may form adduct ions that are only transiently stable (lasting less than a millisecond) at ambient temperature. Moreover, organophosphorus compounds are highly toxic and are commonly used as chemical warfare agents, limiting their practical application in detection technologies.

In some examples, dopants according to the present disclosure may address certain limitations of other types or classes of dopants by forming relatively stronger bonds with target molecules, including illicit drugs such as fentanyl and its analogs. The resulting adduct ions may exhibit significantly prolonged stability, allowing for their analysis using spectrometric techniques (e.g., ion mobility spectrometry). Such dopants may enable reliable detection and identification of target substances, improving both sensitivity and specificity in chemical detection applications.

In some aspects, a method for detecting a chemical substance may include collecting a sample containing a substance of interest and introducing it into a reaction chamber. The method may further include mixing the sample with at least one dopant agent within the reaction chamber, where the dopant agent comprises at least one element selected from phosphorus (P), sulfur(S), or arsenic (As), where the element is: (1) covalently bonded to oxygen via a double bond (E═O); and (2) covalently bonded to nitrogen (E—NR¹R²), where E represents P, S, or As, and R¹ and R² are each independently selected from hydrogen, an alkyl group, an aryl group, a substituted alkyl group, or a substituted aryl group. The method may further include utilizing an ionization source to generate an adduct ion formed from the interaction between the analyte and the dopant agent, where the adduct ion comprises the dopant and the substance of interest. The method may further include performing spectrometric analysis on the adduct ion and identifying the substance of interest based on the acquired spectrometric data.

In some aspects, a substance detection system may include a reaction chamber housing that defines an internal reaction chamber. The system may further include a sample supply system in fluid communication with the reaction chamber, configured to introduce at least a portion of a sample containing a substance of interest. Additionally, the system may include a dopant delivery system in fluid communication with the reaction chamber, configured to introduce at least one dopant agent. The dopant agent may include at least one element selected from phosphorus (P), sulfur(S), or arsenic (As), where the element is: (1) covalently bonded to oxygen via a double bond (E═O); and (2) covalently bonded to nitrogen (E—NR¹R²), where E represents P, S, or As, and R¹ and R² are each independently selected from hydrogen, an alkyl group, an aryl group, a substituted alkyl group, or a substituted aryl group. The system may further include an ionization source coupled to the reaction chamber, configured to facilitate the formation of an adduct ion between the substance of interest and the dopant agent. Additionally, the system may include a spectrometric analysis device in fluid communication with the reaction chamber, configured to analyze the adduct ion. The system may further include a processor configured to process the acquired spectrometric data and identify the substance of interest based on the analysis results.

Certain organophosphorus compounds may be used as dopants for the detection of secondary and tertiary amines, and such detection may be facilitated through the formation of proton-bound adduct ions. However, certain compounds may result in formation of such adduct ions exclusively with organophosphorus compounds comprising a P(═O) bond, such as dimethyl methyl phosphonate (DMMP), diisopropyl methyl phosphonate (DIMP), tributyl phosphate (TBP), triethyl phosphate (TEP), and triphenyl phosphate (TPhP). Organophosphorus compounds such as organophosphonates may form a single hydrogen bond with secondary and tertiary amines, resulting in a weak interaction, which is insufficient to maintain stability in ion mobility spectrometry (IMS). Such proton-bound heterodimers may be observed in IMS under conditions where the drift tube temperature is maintained below 0° C. However, such a configuration is impractical given that modern IMS-based trace detection systems operate with drift tube temperatures around 200° C., and such techniques using certain organophosphorus compounds may not be suitable for IMS applications.

In some examples, techniques according to the present disclosure may be used to detect a class of chemical substances containing at least one amine (E—NR¹R²) or amide (E—C(═O)NR¹R₂) bond using particular dopants. In some examples, a dopant includes at least one element selected from phosphorus (P), sulfur(S), or arsenic (As), where the selected element (E) is: (1) covalently bonded to oxygen via a double bond (E═O) and (2) covalently bonded to nitrogen (E—NR¹R²). These specific structural features may enable the formation of multiple stabilizing interactions with a wide range of target analytes, including fentanyl and other chemicals containing amine and amide functionalities, thereby enhancing detection capabilities in analytical techniques such as Ion Mobility Spectrometry (IMS) and Mass Spectrometry (MS).

Mechanisms of interaction with chemicals containing amine and amide bonds may include hydrogen bond formation. For example, the E═O group (phosphoryl, sulfinyl, or arsenyl oxygen) serves as a hydrogen bond acceptor, forming stable interactions with the amine groups present in the target analytes. This interaction may enhance the longevity and detectability of the resulting adduct. The E—NR¹R² group acts as a hydrogen bond donor, capable of forming an additional hydrogen bond with either: (a) The carbonyl oxygen (C═O) of amides, providing a stabilizing effect in analytes containing peptide bonds or other amide functionalities; (b) The lone pair of electrons on secondary or tertiary amines, further stabilizing the complex. The presence of at least two hydrogen bonds prevents rapid dissociation, improving the sensitivity and specificity of detection.

Certain mechanisms may involve ion-dipole and electrostatic interactions. When ionized, the disclosed dopants carry a partial or full positive charge, enhancing their interaction with polar functional groups in the target analytes. The tertiary amine (—NR₂) or secondary amine (—NHR) groups in many chemical substances can form strong ion-dipole interactions with the partially negative phosphoryl oxygen (P═O), sulfinyl oxygen (S═O), or arsenyl oxygen (As═O) in the dopant molecule. Similarly, the amide carbonyl (C═O) group, being an electron-rich dipole, interacts with the positively charged dopant, further stabilizing the complex and ensuring prolonged adduct lifetime under detection conditions. These combined hydrogen bonding and ion-dipole interactions create a stable adduct ion that remains intact during Ion Mobility Spectrometry (IMS) and Mass Spectrometry (MS), improving detection reliability.

The ability to form multiple hydrogen bonds and ion-dipole interactions may promote stability of the dopant-analyte complex for detection in IMS, where a prolonged adduct lifetime is critical for maintaining system sensitivity. Unlike dopants that rely on a single hydrogen bond, dopants that can form two or more stabilizing interactions may prevent adduct dissociation at high-temperature IMS conditions (typically ~200°C). The increased stability of these dopant-analyte complexes enables enhanced selectivity and specificity, reducing false negatives and improving trace-level detection of target substances.

In some examples, dopants according to the present disclosure may be used to detect chemicals with amine (—NR¹R²) or amide (—C(═O)NR¹R²) bonds. These include synthetic opioids (fentanyl, sufentanil, remifentanil), amphetamines and methamphetamines, cocaine and derivatives, peptides and small proteins, pharmaceutical compounds, and nitrogen-based chemical warfare agents (vesicants, nerve agents). In some examples, dopants characterized by the presence of an E═O bond and an E—NR¹R² bond (where E represents phosphorus, sulfur, or arsenic), provides a superior method for detecting a wide range of chemicals containing amine and amide bonds. The ability to form multiple hydrogen bonds and ion-dipole interactions enhances the stability and detectability of the resulting adduct ions, ensuring reliable detection in IMS, MS, and other analytical systems. This approach enables more sensitive, selective, and robust detection of illicit drugs, pharmaceuticals, and chemical warfare agents in real-world applications.

In some examples, diethyl phosphoramidate (DEPA) may be used as a dopant or reagent, that interacts or reacts with a target species (e.g., an analyte) to generate a product species that may be more accurately and sensitively detected by spectrometric devices including IMS and MS compared to the target species itself. The CAS number of diethyl phosphoramidate (DEPA) is 1068-21-9, and the chemical formula is C₄H₁₂NO₃P. The toxicity of DEPA is very low. Positively ionized diethyl phosphoramidate [DEPA]⁺ can form two hydrogen bonds with fentanyl, primarily through interactions involving the P═O group as a hydrogen bond acceptor and the P—NH₂ group as a hydrogen bond donor. These hydrogen bonds contribute to the stability of the resulting adduct ion, facilitating its detection in Ion Mobility Spectrometry (IMS) or Mass Spectrometry (MS).

In addition to hydrogen bonding, ion-dipole interactions play a significant role in stabilizing the [DEPA+fentanyl+H]⁺ complex. Since diethyl phosphoramidate is positively ionized, it introduces an electrostatic component that enhances interaction with fentanyl's polar functional groups. The tertiary amine in fentanyl, which can carry a partial or full positive charge depending on protonation state, can interact with the partially negative phosphoryl oxygen (P═O) via ion-dipole attraction. Likewise, the amide carbonyl (C═O) in fentanyl, being a strong dipole, can further interact with the positively charged phosphorus center, adding an additional layer of stabilization.

These combined hydrogen bonding and ion-dipole interactions significantly enhance the stability and longevity of the [DEPA+fentanyl+H]⁺ ion adduct, making it detectable under conditions where weaker, single hydrogen-bonded adducts might dissociate. This increased stability is particularly advantageous for IMS applications, where a prolonged adduct lifetime is essential for maintaining system sensitivity and selectivity.

In some examples, tert-Butylsulfinamide (TBSA) may be used as a dopant or reagent, that interacts or reacts with a target species to generate a product species that may be more accurately and sensitively detected by spectrometric devices. The CAS number of TBSA is 146374-27-8, and the chemical formula is C₄H₁₁NOS. The UIPAC name is 2-methylpropane-2-sulfinamide.

In some examples, 4-Methylbenzenesulfinamide (MBSA) may be used as a dopant or reagent, that interacts or reacts with a target species to generate a product species that may be more accurately and sensitively detected by spectrometric devices. The CAS number of MBSA is 6873-55-8, and the chemical formula is C₇H₉NOS. The UIPAC name is 4-methylbenzenesulfinamide. The toxicity of MBSA is very low.

Both tert-butylsulfinamide and 4-methylbenzenesulfinamide form multiple hydrogen bonds and ion-dipole interactions with fentanyl and similar substances, enhancing adduct stability for reliable detection. The bulky tert-butyl group in tert-butylsulfinamide provides selectivity, while 4-methylbenzenesulfinamide's aromatic ring enhances additional π-stacking interactions. These features make sulfinamide-based dopants highly effective for IMS, MS, and other detection systems, improving the sensitivity, specificity, and robustness of fentanyl and related substance detection.

Certain detection mechanisms may involve hydrogen bond formation. The S═O group in sulfinamides acts as a hydrogen bond acceptor, interacting with the amide (—C(═O)NR¹R²) or amine (—N R¹R²) groups in fentanyl-related substances. The amine group in sulfinamides serves as a hydrogen bond donor, forming an additional hydrogen bond with the carbonyl oxygen (C═O) of amides or the lone pair of a tertiary amine in fentanyl-like molecules. This dual hydrogen bonding effect leads to strong adduct formation, preventing rapid dissociation under ionization conditions.

Certain detection mechanisms may involve ion-dipole and electrostatic stabilization. Sulfinamides can be ionized, especially in proton-rich environments such as Electrospray Ionization (ESI) or Chemical Ionization (CI), leading to enhanced ion-dipole interactions. Fentanyl's tertiary amine (—NR₂), often protonated in detection systems, interacts electrostatically with the partially negative sulfinyl oxygen (S═O), enhancing adduct stability. The amide carbonyl (C═O) in fentanyl or similar molecules forms additional dipole-dipole interactions with the positively charged sulfinamide nitrogen, contributing to overall complex stabilization.

In some examples, example methods may include mixing a sample with at least one dopant within a reaction chamber to facilitate the formation of detectable adduct ions. In some examples, the dopant may be pre-applied to a sampling swab, where it is thermally desorbed along with the collected sample during analysis. In some examples, a method may further include ionizing an analyte (e.g., in the sample) in the presence of at least one dopant to generate an adduct ion comprising the dopant and a dissociated ion derived from the analyte. The ionization source employed in the method can be any suitable ionization system that enables the operation of the disclosed methods and systems, including, but not limited to, a radioactive ionization source, electrospray ionization (ESI), atmospheric pressure chemical ionization (APCI), atmospheric pressure photoionization (APPI), atmospheric pressure glow discharge (APGD), direct analysis in real time (DART), and atmospheric pressure dielectric barrier discharge (APDBD). In certain embodiments, the ionization source includes at least one of APCI, APPI, ESI, or DART. Additionally, in some embodiments, the system is configured to operate at sub-atmospheric pressures, utilizing ionization sources such as chemical ionization (CI), photoionization (PI), glow discharge (GD), or dielectric barrier discharge (DBD).

In some examples, a substance detection system is provided. The substance detection system may include a reaction chamber housing that defines a reaction chamber therein; a sample supply system operatively coupled in flow communication with the reaction chamber, the sample supply system being configured to introduce at least a portion of a sample of interest into the reaction chamber; a dopant system operatively coupled in flow communication with the reaction chamber, the dopant system being configured to deliver at least one dopant into the reaction chamber. The system may further include an ionization source operatively coupled with the reaction chamber, the ionization source being configured to promote the formation of an adduct between the sample of interest and the at least one dopant. The system may further include a spectrometric analysis device operatively coupled in flow communication with the reaction chamber, the spectrometric analysis device being configured to perform spectrometric analysis of the adduct. The system may further a processor operatively coupled with the spectrometric analysis device. The processor may be configured to identify at least one substance of interest based on comparative spectrometric data.

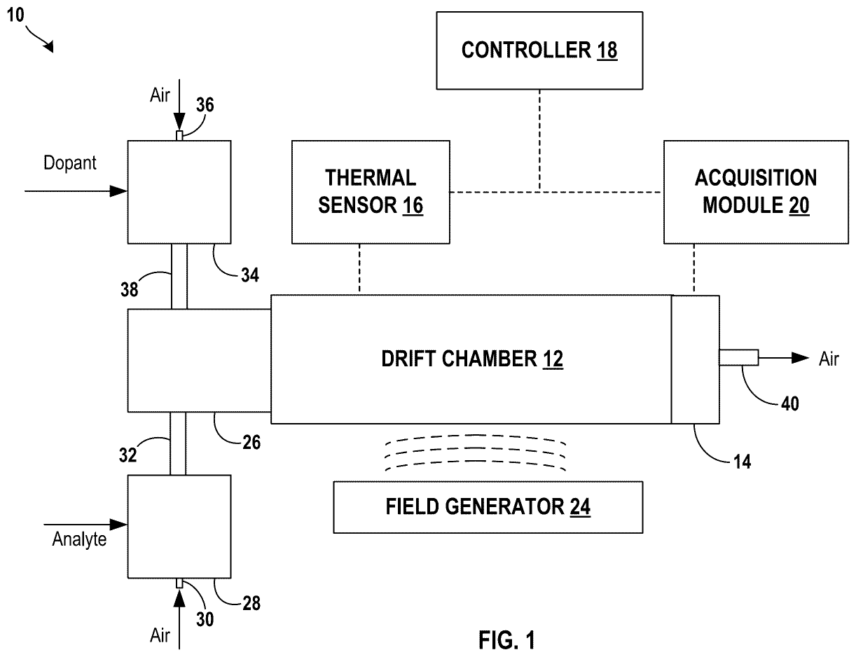

FIG. 1 is a block diagram illustrating an example system 10 for ion mobility spectrometry, in accordance with one or more aspects of this disclosure. System 10 may include a drift chamber 12, an ion detector 14, a thermal sensor 16, and a controller 18. Drift chamber 12 is configured to receive an ionized sample and an ionized dopant. Drift chamber 12 may include a housing. The housing may include at least one metal or alloy, or any other suitable material. The housing may be cylindrical or tubular (so that the drift chamber is a drift tube), or have any other suitable shape.

The ion detector 14 is configured to generate a drift signal in response to detecting drifted ions at a predetermined position along drift chamber 12. The ion detector 14 may be positioned at or near the predetermined position. In some examples, ion detector 14 is positioned at or adjacent an end or a face of drift chamber 12. In some examples, ion detector 14 includes an ion collector configured to attract the ionized sample and the ionized dopant. For example, the ion collector may include a Faraday cup.

Thermal sensor 16 is configured to sense a temperature of drift chamber 12 (for example, within an interior drift region of drift chamber 12) and generate a temperature signal indicative of the temperature of drift chamber 12. Thermal sensor 16 may include a thermocouple, a resistance temperature detector (RTD) sensor, an infrared (IR) sensor, or any other suitable sensor. Thermal sensor 16 (or a probe thereof) may be positioned to sense a temperature within an interior of drift chamber 12, at a surface of drift chamber 12, an area near drift chamber 12, or another location whose temperature may be correlated to a temperature within a drift region within drift chamber 12. In some examples, system 10 includes a plurality of thermal sensors 16, and a temperature of a drift region or an interior of drift chamber 12 may be determined based on temperature signals received from the plurality of thermal sensors 16.

In some examples, drift chamber 12 is not actively heated, and the temperature within drift chamber 12 is substantially the same as room temperature or ambient temperature. Without being bound by theory, operation at ambient temperature versus controlled higher temperatures may preserve the stability of thermally labile compounds, enhancing their detection performance.

In other examples, system 10 further includes a chamber heater (not shown) configured to heat drift chamber 12 to a predetermined temperature. In some examples, drift chamber 12 is maintained at a substantially constant target temperature. In some examples, the chamber heater provides a thermal gradient along a portion of drift chamber 12, for example, along a length of drift chamber 12.

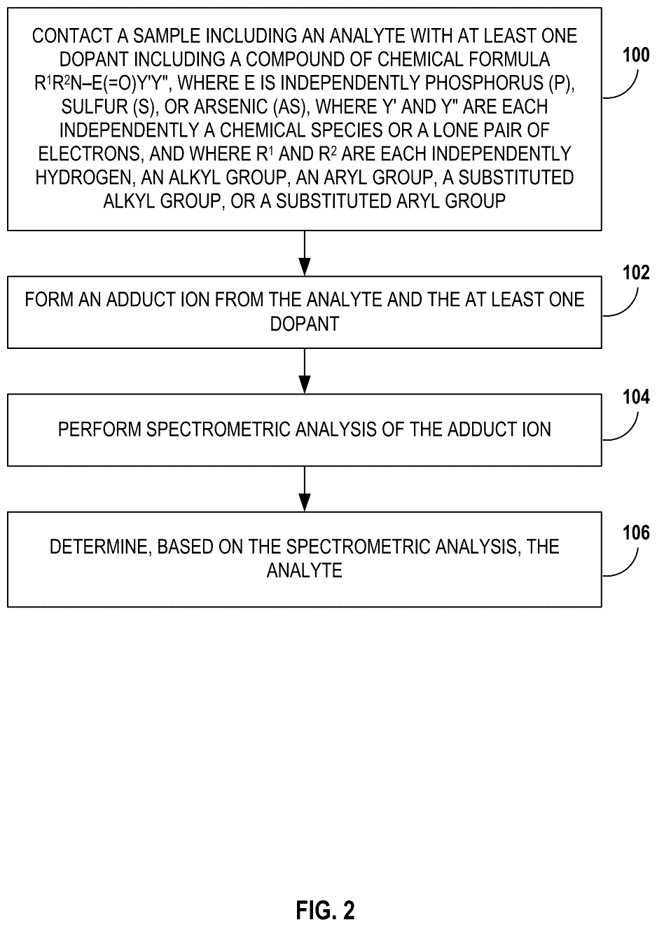

Controller 18 may include a computing device (for example, as described with reference to FIG. 2) or processing circuitry. The processing circuitry may be configured to determine, based on the drift signal, a sample drift time. The processing circuitry may be further configured to detect a target species (e.g., an analyte) in the sample (e.g., the ionized sample) by comparing the sample drift time with the target drift time. For example, the processing circuitry may compare the sample drift time with the expected drift time for the target species (or with a plurality of expected drift times for different target species) to detect the target species (or one species of the plurality of target species) in the sample. For example, if the sample drift time and the expected drift time are equal, or approximately equal within a predetermined tolerance range, the processing circuitry may determine that the sample includes the target species. If the sample drift time and the expected drift time differ in value, for example, beyond the predetermined tolerance range, the processing circuitry may determine that the target species is absent in the sample.

In some examples, the processing circuitry is further configured to determine a presence of the target species in response to determining that the sample drift time is within a predetermined drift time range including the target drift time. In some examples, the predetermined drift time range is ±10% of the target drift time, or ±5% of the target drift time, or ±3% of the target drift time, or ±1% of the target drift time. The predetermined drift time range may be symmetric or asymmetric about the target drift time. For example, if manufacturing or measurement tolerances typically result in an underestimate of the target drift time, the drift time range may have a lower bound that is relatively more distant from the target drift time compared to an upper bound.

Any suitable analyte may be detected by system 10, for example, using an appropriate dopant for the analyte. In some examples, the analyte includes a narcotic. The narcotic may include at least one of heroin, cocaine, tetrahydrocannabinol (THC), cannabidiol (CBD), xylazine, nitazenes, fentanyl, norfentanyl, carfentanil, acetyl fentanyl, acrylfentanyl, or a fentanyl analog. In some examples, the analyte includes a chemical weapon substance. In some examples, the chemical weapon substance includes at least one of tabun, sarin, soman, VX nerve agent, mustard gas, or a mustard gas derivative.

The processing circuitry may be further configured to generate an output indicative of the presence of the analyte (or some species representative of the analyte, e.g., a fragment or derivative thereof). For example, system 10 may include an output device (not shown in FIG. 1), and controller 18 may be configured to send an output signal to the output device, which may display or generate an output in response to the output signal. For example, the output may include at least one of a digital signal, an audio signal, or a display signal. In some examples, one or more of the digital signal, the audio signal, or the display signal may be indicative of an identity of the analyte, presence or absence of the analyte, or a concentration of the analyte. For example, the display signal may be configured to cause the output device to display an alphanumeric or graphical output indicative of the identity or concentration of the analyte. The display may include a table of potential analytes, and indicate whether one or more of the potential analytes are present or absent, or respective concentration of potential analytes detected in the sample. The audio signal may be configured to cause the output device to generate an alert or a beep sound (e.g., a beep tone) indicative of a presence of the analyte. In some examples, the audio signal may be configured to cause the output device to generate a first alert sound indicative of a presence of the analyte, and a second alert sound indicative of an absence of the analyte. The digital signal may be configured to be transmitted by a network to another device, for example, for storage on a storage device, or display on an output device, or for interfacing with an application hosted by another device (e.g., a mobile device).

System 10 may further include a data acquisition module 20 coupled to the ion detector 14, with controller 18 being communicatively coupled to data acquisition module 20. Data acquisition module 20 is configured to generate data indicative of ions detected by ion detector 14. For example, ion detector 14 may generate an analog signal, while data acquisition module 20 may generate a digital signal based on the analog signal received from ion detector 14. In other examples, controller 18 itself may include a data acquisition module, or otherwise acquire a drift signal from ion detector 14. Controller 18 may be used to perform spectrometric analyses of data imported from data acquisition module 20 (or ultimately from ion detector 14). Alternatively, or in addition, controller 18 may be used to control the operation of system 10, for example, by controlling one or more of drift chamber 12, ion detector 14, data acquisition module 20, or any other apparatus associated with or a component of system 10.

System 10 may further include a field generator 24 configured to generate an electric field along drift chamber 12. In some examples, the electric field promotes drifting of the ionized sample and the ionized dopant along the drift chamber toward the ion detector 14.

System 10 may further include an ionization chamber 26 configured to ionize a sample into the ionized sample and a dopant into the ionized dopant. Ionization chamber 26 may include an energy source, for example, an arc, a corona, or a plasma, or some other ionization source. In some such examples, drift chamber 12 is between ion detector 14 and ionization chamber 26. In some examples, ionization chamber 26 may face or be coupled to a first face or end of drift chamber 12, while ion detector 14 may face or be coupled to a second face or end of drift chamber 12 opposing the first face or end. In some examples, drift chamber 12, ion detector 14, and ionization chamber 26 may be formed as separate partitions or compartments within a single housing. In some examples, drift chamber 12, ion detector 14, and ionization chamber 26 may be mechanically coupled to secured to each other. In some such examples, drift chamber 12, ion detector 14, and ionization chamber 26 may be removably coupled. For example, ion detector 14 or ionization chamber 26 may be removable or separable from drift chamber 12 to facilitate maintenance, cleaning, or repair of one or more of drift chamber 12, ion detector 14, or ionization chamber 26.

The sample volume introduced into ionization chamber 26 may be generated by fluidization of an analyte sample. The analyte sample may include a solid, liquid, gel, suspension, or a composition in any suitable form including an analyte. In some such examples, system 10 further includes a sample chamber 28 configured to fluidize an analyte sample to generate the sample volume. For example, sample chamber 28 may include or define a sample air inlet 30 configured to introduce air to fluidize the analyte sample. In some examples, sample air inlet 30 includes a nozzle. Air (or another fluidizing carrier gas or gaseous mixture) may be drawn into sample air inlet 30 by a pressure differential or a motive element, and may fluidize the analyte sample into the sample volume. The fluidization may include vaporization or aerosolization. Thus, the sample volume may include particles or droplets of the analyte sample suspended in air, or vaporized or gas molecules of the analyte sample. In some examples, system 10 further includes a sample line 32 fluidly coupling sample chamber 28 to ionization chamber 26 and configured to introduce the sample volume into ionization chamber 28. In some examples, sample chamber 28 includes a heating system configured to heat sample chamber 28 (and thus, the sample volume) to a predetermined temperature.

System 10 may include another chamber, for example, to introduce a dopant into ionization chamber 26. In some examples, system 10 further includes a dopant chamber 34 configured to fluidize a dopant composition to generate the dopant. Any suitable dopant composition may be used.

Dopant chamber 34 may include or define a dopant air inlet 36 configured to introduce air to fluidize the dopant composition into the dopant. In some examples, dopant air inlet 36 includes a nozzle. Air (or another fluidizing carrier gas or gaseous mixture) may be drawn into dopant air inlet 36 by a pressure differential or a motive element, and may fluidize the dopant composition to form a volume of the dopant. Thus, the dopant volume may include particles or droplets of the reagent composition suspended in air, or vaporized or gas molecules of the reagent composition (also referred to as “dopant”). In some examples, system 10 further includes a reagent line 38 fluidly coupling dopant chamber 34 to ionization chamber 26 and configured to introduce the dopant into ionization chamber 26. In some examples, dopant chamber 34 includes a heating system configured to heat dopant chamber 34 (and thus, the dopant) to a predetermined temperature.

The dopant is configured to interact with or react with the analyte or the ionized analyte, for example, to form an adduct ion. Thus, drift chamber 12 may be configured to receive the adduct ion including an ionized product of at least one dopant and a sample (e.g., including an analyte). Any suitable dopant composition may be used to form the at least one dopant. In some examples, the dopant composition or the at least one dopant includes a compound of Chemical Formula 1:

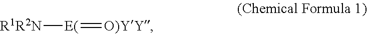

where E is independently phosphorus (P), sulfur(S), or arsenic (As), where Y′ and Y″ are each independently a chemical species or a lone pair of electrons, and where R¹ and R² are each independently selected from hydrogen, an alkyl group, an aryl group, a substituted alkyl group, or a substituted aryl group

In some examples, E is P, and wherein the at least one compound has Chemical Formula 2:

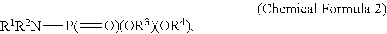

where R³ and R⁴ are each independently selected from hydrogen, an alkyl group, an aryl group, a substituted alkyl group, or a substituted aryl group.

In some examples, E is P, and the at least one compound includes a phosphoramidic acid having Chemical Formula 3:

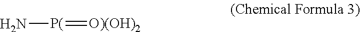

In some examples, E is P, and the at least one compound includes a nucleotide phosphoramidate.

In some examples, E is S, and the at least one compound includes a sulfinamide, having Chemical Formula 4:

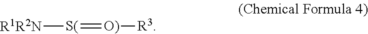

In some examples, E is S, and the at least one compound comprises a sulfonamide, having Chemical Formula 5:

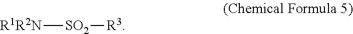

In some examples, E is S, and the at least one compound comprises a sulfamate, having Chemical Formula 6:

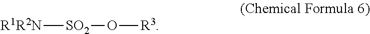

In some examples, the at least one compound has Chemical Formula 7:

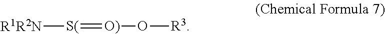

In some examples, the at least one dopant includes one or more of diethyl phosphoramidate (DEPA), tert-Butylsulfinamide (TBSA), or 4-Methylbenzenesulfinamide (MBSA).

Air or another carrier fluid introduced into drift chamber 12, for example, via ionization chamber 26, may flow along drift chamber 12, carrying ions (e.g., adduct ions) along drift chamber 12, and exit drift chamber 12. For example, drift chamber 12 may further include an air outlet 40 configured to allow a stream of air to flow along and depart drift chamber 12. In some examples, air may flow through drift chamber 12 into ion detector 14, and ion detector 14 may include air outlet 40. Air outlet 40 may include or be coupled to an exhaust device or system, for example, a pump or a fan, to promote exhausting or removal of air from drift chamber 12.

Thus, system 10 may be used to perform IMS using a predetermined dopant.

In some examples, a portable spectrometry device may include a handheld housing, the handheld housing including ion mobility spectrometry system 10. For example, the handheld housing may be dimensioned and configured to be carried and operated by hand. In some examples, the handheld housing includes a sample inlet to introduce a sample, and a dopant inlet to introduce a dopant. In some examples, the portable spectrometry device includes a benchtop housing including ion mobility spectrometry system 10.

FIG. 2 is a flow diagram illustrating an example method for detecting an analyte in a sample, in accordance with one or more aspects of this disclosure. While the method of FIG. 2 is described with reference to system 10 of FIG. 1, the method of FIG. 2 may be implemented using any suitable system.

In some examples, the method of FIG. 2 includes contacting a sample including an analyte with at least one dopant (100). For example, the sample and the dopant may be introduced separately or together in a chamber. The at least one dopant may include a compound of Chemical Formula 1. In some examples, the at least one dopant includes a compound of one of more of Chemical Formula 2, Chemical Formula 3, Chemical Formula 4, Chemical Formula 5, Chemical Formula 6, or Chemical Formula 7, or a derivative or analog thereof.

The method may further include forming an adduct ion from the analyte and the at least one dopant (102). In some examples, forming the adduct ion (102) includes ionizing the analyte (e.g., in the sample) and the at least one dopant to generate the adduct ion. For example, the analyte and the dopant may be ionized in ionization chamber 26 to generate the ionized analyte and the ionized dopant, respectively. In some such examples, the method may further include one or both of fluidizing an analyte composition to generate the analyte or fluidizing a dopant composition to generate the dopant. For example, the analyte composition may be fluidized in sample chamber 28, and the dopant composition may be fluidized in dopant chamber 34, to generate the analyte and the dopant volume, respectively, to be introduced into ionization chamber 26.

The adduct ion may flow along drift chamber 12, for example, toward ion detector 14, at a respective mobility. In some examples, the method further includes generating an electric field along drift chamber 12, for example, along at least a portion of drift chamber 12. The mobilities of the ionized sample and the ionized dopant may be independently influenced by the electric field. For example, the electric field may have a field strength configured to sufficiently separate the ionized sample and the ionized dopant by inducing a difference in their respective mobilities.

Drift chamber 12 may be maintained at any suitable temperature, for example, ambient or room temperature, lower than ambient, or greater than ambient. In some examples, drift chamber 12 is at room temperature. In some examples, drift chamber 12 is at a temperature of 150° C. or less. In some such examples, drift chamber 12 is at a temperature of 80° C. or less. Drift chamber 12 may be at a temperature of at least 20° C., at least 25° C., at least 30° C., at least 35° C., at least 40° C., at least 45° C., at least 50° C., at least 60° C., at least 70° C., at least 80° C., at least 90° C., at least 100° C., at least 110° C., at least 120° C., at least 125° C., at least 130° C., at least 135° C., or at least 140° C. Drift chamber 12 may be at a temperature of 150° C. or less, 140° C. or less, 135° C. or less, 125° C. or less, 120° C. or less, 115° C. or less, 110° C. or less, 105° C. or less, 100° C. or less, 90° C. or less, 80° C. or less, 70° C. or less, 60° C. or less, 50° C. or less, 45° C. or less, 40° C. or less, 35° C. or less, 30° C. or less, or 25° C. or less.

The sample (or ultimately, the analyte sample) may include at least one of an explosive, a narcotic, a chemical warfare agent, a pesticide, a toxic industrial chemical, or a pharmaceutical trace contaminant. In some examples, the analyte includes a narcotic. For example, the narcotic may include at least one of heroin, cocaine, tetrahydrocannabinol (THC), cannabidiol (CBD), xylazine, nitazenes, fentanyl, norfentanyl, carfentanil, acetyl fentanyl, acrylfentanyl, or a fentanyl analog. In some examples, the analyte includes a chemical weapon substance. For example, the chemical weapon substance may include at least one of tabun, sarin, soman, VX nerve agent, mustard gas, or a mustard gas derivative.

The technique may further include performing spectrometric analysis of the adduct ion (104). In some examples, the performing spectrometric analysis (104) includes performing mass spectrometry. In some examples, the performing spectrometric analysis (104) includes performing ion mobility spectrometry. For example, the method may further include introducing an adduct ion including an ionized product of the at least one dopant and the analyte in drift chamber 12. The method may further include generating, by ion detector 14, a drift signal in response to detecting drifted ions (e.g., adduct ions) at a predetermined position along drift chamber 12. The method may further include, by the processing circuitry (or controller 18), determining, based on the drift signal, a sample drift time. The method may further include, by the processing circuitry (or controller 18), detecting the analyte in the ionized sample by comparing the sample drift time with a target drift time. For example, the processing circuitry (or controller 18) may mathematically compare the values of the sample drift time and the target drift time (or a plurality of target drift times). The plurality of target drift times may be associated with a respective plurality of candidate analytes. The closest drift time of the plurality of drift times to the sample drift time may be indicative of the analyte present in the sample.

The method may further include generating, by thermal sensor 16, a temperature signal indicative of a temperature of drift chamber 12.

Determining the analyte (106) may include determining, by the processing circuitry (or controller 18), a presence of the analyte in response to determining that the sample drift time is within a predetermined drift time range including the target drift time. For example, the processing circuitry (or controller 18) may determine that the analyte is present in the sample if the value of the sample drift is equal, or approximately equal within a predetermined tolerance range, to the target drift time. In some examples, the processing circuitry (or controller 18) may determine that a particular analyte of a plurality of analytes is present in the sample if the value of the sample drift time is equal, or approximately equal within a predetermined tolerance range, to the target drift time associated with the particular target analyte. For example, each analyte of the plurality of analytes may be associated with different expected drift times, and only one of the expected drift times may substantially match the sample drift time.

The method may further include, by the processing circuitry (or controller 18), generating an output indicative of the presence of the analyte. The output may include at least one of a digital signal, an audio signal, or a display signal.

Thus, an operator may be alerted to the presence or absence of one or more analyte in the sample, for example, by the output.

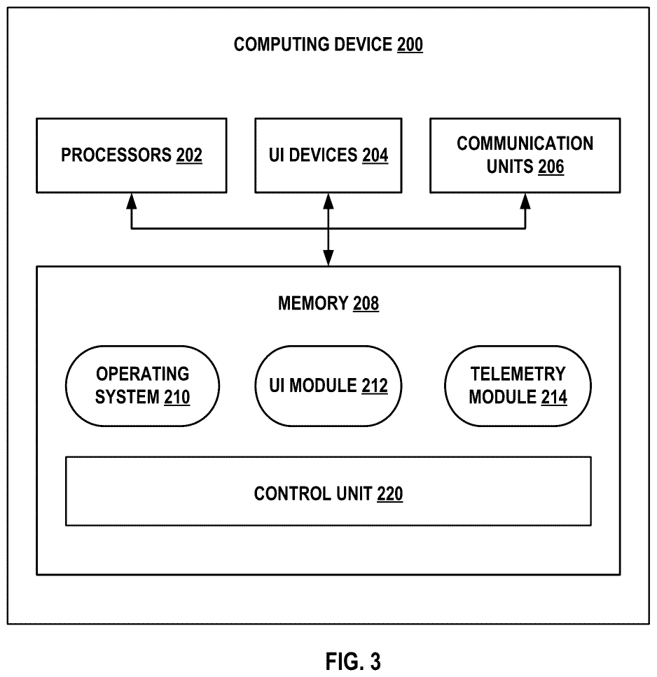

FIG. 3 is a block diagram illustrating an example computing device 200 configured to control a system for ion mobility spectrometry, in accordance with one or more aspects of this disclosure. In some examples, computing device 200 may be an example instance of controller 18 of FIG. 1.

As shown in the example of FIG. 3, computing device 200 includes one or more processors 202, one or more user interface (UI) devices 204, one or more communication units 206, and one or more memory units 208. Memory 208 of computing device 200 includes operating system 210, UI module 212, telemetry module 214, and control unit 220, which are executable by processors 202. Each of the components, units or modules of computing device 200 are coupled (physically, communicatively, and/or operatively) using communication channels for inter-component communications. In some examples, the communication channels may include a system bus, a network connection, an inter-process communication data structure, or any other method for communicating data.

Processors 202, in one example, may comprise one or more processors that are configured to implement functionality and/or process instructions for execution within computing device 200. For example, processors 202 may be capable of processing instructions stored by memory 208. Processors 202 may include, for example, microprocessors, digital signal processors (DSPs), application specific integrated circuits (ASICs), field-programmable gate array (FPGAs), or equivalent discrete or integrated logic circuitry, or a combination of any of the foregoing devices or circuitry.

Memory 208 may be configured to store information within computing device 200 during operation. Memory 208 may include a computer-readable storage medium or computer-readable storage device. In some examples, memory 208 includes one or more of a short-term memory or a long-term memory. Memory 208 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 208 is used to store program instructions for execution by processors 202. Memory 208 may be used by software or applications running on computing device 200 (e.g., control unit 220) to temporarily store information during program execution.

Computing device 200 may utilize communication units 206 to communicate with external devices via one or more networks or via wireless signals. Communication units 206 may be network interfaces, such as Ethernet interfaces, optical transceivers, radio frequency (RF) transceivers, or any other type of devices that can send and receive information. Other examples of interfaces may include Wi-Fi, NFC, or Bluetooth radios. In some examples, computing device 200 utilizes communication units 206 to wirelessly communicate with one or more external devices, such as ion detector 14, thermal sensor 16, controller 18, or acquisition module 20 from FIG. 1.

UI devices 204 may be configured to operate as both input devices and output devices. For example, UI devices 204 may be configured to receive tactile, audio, or visual input from a user of computing device 200. In addition to receiving input from a user, UI devices 204 may be configured to provide output to a user using tactile, audio, or video stimuli. In one example, UI devices 204 may be configured to output content such as a GUI for display at a display device. UI devices 204 may include a presence-sensitive display that displays a GUI and receives input from a user using capacitive, inductive, and/or optical detection at or near the presence sensitive display.

Other examples of UI devices 204 include a mouse, a keyboard, a voice responsive system, video camera, microphone or any other type of device for detecting a command from a user, or a sound card, a video graphics adapter card, or any other type of device for converting a signal into an appropriate form understandable to humans or machines. Additional examples UI devices 204 include a speaker, a cathode ray tube (CRT) monitor, a liquid crystal display (LCD), organic light emitting diode (OLED), or any other type of device that can generate intelligible output to a user.

Operating system 210 controls the operation of components of computing device 200. For example, operating system 210, in one example, facilitates the communication of UI module 212, telemetry module 214, and control unit 220 with processors 202, UI devices 204, communication units 206, and memory 208. UI module 212, telemetry module 214, and control unit 220 may each include program instructions and/or data stored in memory 208 that are executable by processors 202. For example, control unit 220 may include instructions that cause computing device 200 to perform one or more of the techniques described in this disclosure.

Computing device 200 may include additional components that, for clarity, are not shown in FIG. 3. For example, computing device 200 may include a battery to provide power to the components of computing device 200. Similarly, the components of computing device 200 shown in FIG. 3 may not be necessary in every example of computing device 200.

In the example illustrated in FIG. 3, control unit 220 may be configured to control system 10 and/or any of its components, for example ion detector 14, thermal sensor 16, controller 18, or acquisition module 20, and/or any other hardware or component of system 10, for example, a fan or pump to move a volume or stream to or along drift chamber 12, and the like. In some examples, control unit 220 may be configured to detect the analyte, for example, a presence, absence, concentration or amount of analyte in the analyte sample, for example, based on data received from controller 18 and/or telemetry module 214. In some examples, control unit 220 may cause computing device 200 and/or processors 202 to execute one, more, or all portions of example methods described in the present disclosure, for example, example methods described with reference to FIG. 2.

In some examples, a sampling device may include at least one dopant according to the present disclosure, for example, according to any of Chemical Formula 1 to 7, or their derivatives or analogs. In some examples, the sampling device includes a sampling swab pre-treated with a dopant agent (e.g., a solid or liquid composition including the dopant). The sampling swab may include a handle, and a swab portion configured to carry the sample. In some examples, the swab portion is configured to at least partially absorb the sample. Contacting a sample with the swab portion may thus result in contact between the dopant and the sample, and the sample may be retained on the swab portion with the dopant. The swab portion may then be further processed (e.g., using spectrometric analysis) to determine the analyte in the sample.

### EXAMPLES

The following examples illustrate various example aspects according to the present disclosure. An ion mobility spectrometer with room temperature drift tube was used in EXAMPLES 1 to 4. Each substance was deposited and dried on a PTFE-coated fiberglass swab, inserted into a thermal desorber, and volatilized into an ionization source using Atmospheric Pressure Ionization Source. Ionization was performed in positive mode, generating and detecting positive ions. A dopant agent was co-deposited with the substance on the sampling swab.

### Example 1

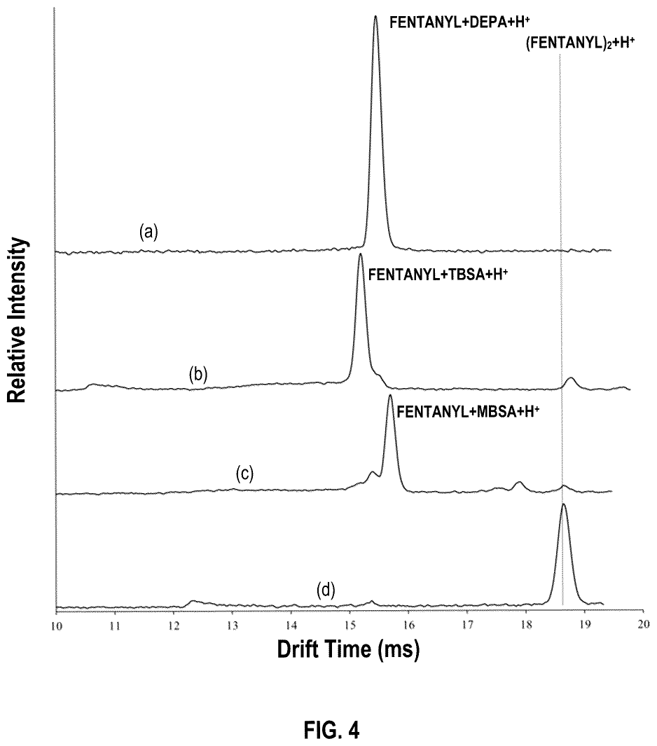

Fentanyl was selected as the substance of interest. A trial amount of fentanyl was 100 ng, and the amount of deposited dopant agent was 1 μg in each trial run. Plasmagrams were obtained for each separate trial run. The trial runs were conducted for the following combinations: (a) fentanyl and diethyl phosphoramidate (DEPA) as a dopant agent; (b) fentanyl and tert-Butylsulfinamide (TBSA) as a dopant agent; (c) fentanyl and 4-Methylbenzenesulfinamide (MBSA) as a dopant agent; and (d) fentanyl alone without any dopant agent. The plasmagrams are presented in FIG. 4. FIG. 4 is a plot illustrating example ion mobility plasmagrams of samples including acrylfentanyl in presence and absence of a dopant, in accordance with one or more aspects of this disclosure. As shown in FIG. 4, at room temperature, fentanyl forms a distinct protonated fentanyl dimer peak. When the dopant agent was present in the ionization source serving here as a reaction chamber, fentanyl molecules formed adduct ions with the dopant agents: [fentanyl+DEPA+H]⁺, [fentanyl+TBSA+H]⁺, and [fentanyl+MBSA+H]⁺.

### Example 2

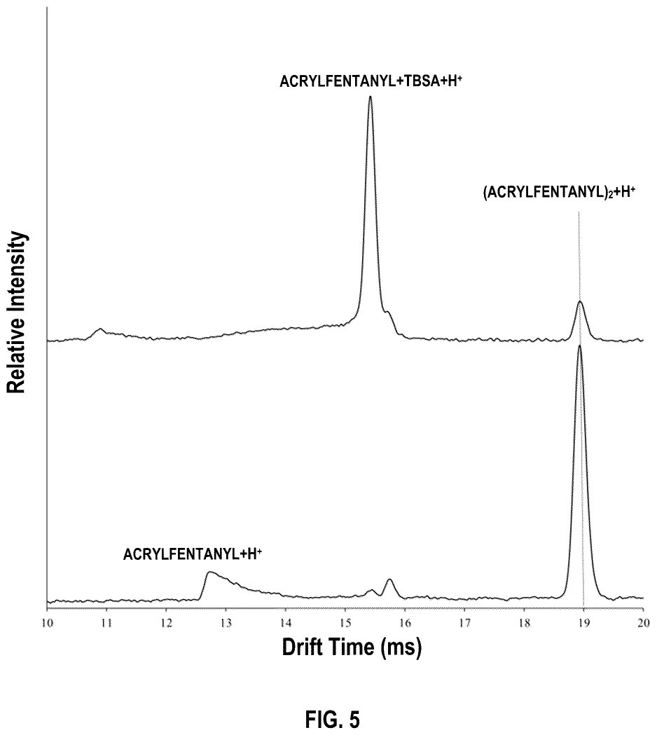

Acrylfentanyl was chosen as the substance of interest. A trial amount of acrylfentanyl was 100 ng, and the amount of deposited TBSA dopant agent was 1 μg. FIG. 5 is a plot illustrating example ion mobility plasmagrams of samples including acrylfentanyl in presence and absence of a dopant, in accordance with one or more aspects of this disclosure. Two peaks in the lower curve in FIG. 5 correspond to acrylfentanyl monomer and dimer ions. The upper curve shows an ion mobility plasmagram of acrylfentanyl with TBSA as a dopant agent. Acrylfentanyl molecules formed adduct ions with TBSA [acrylfentanyl+TBSA+H]⁺.

### Example 3

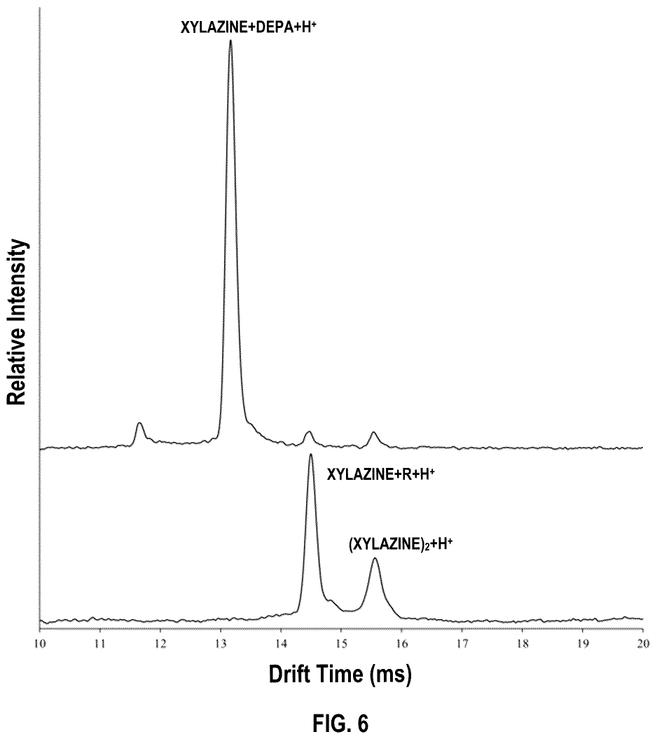

Xylazine was chosen as the substance of interest. A trial amount of xylazine was 1 ug, and the amount of deposited DEPA dopant agent was 5 μg. FIG. 6 is a plot illustrating example ion mobility plasmagrams of samples including xylazine in presence and absence of a dopant, in accordance with one or more aspects of this disclosure. The lower curve in FIG. 6 shows an ion mobility plasmagram of a pure xylazine sample without any dopant. The upper curve in FIG. 6 shows an ion mobility plasmagram of xylazine with DEPA as a dopant agent. Xylazine molecules formed adduct ions with DEPA [xylazine+DEPA+H]⁺.

### Example 4

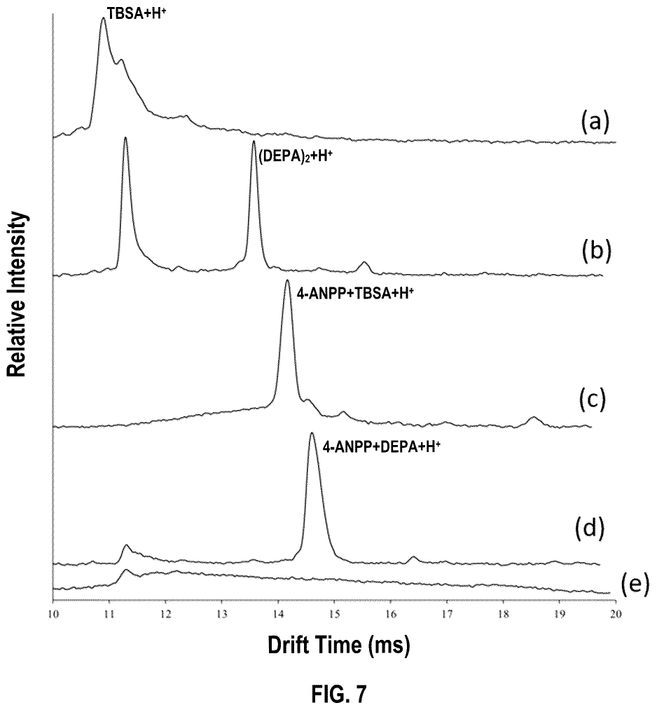

In Example 4, 4-anilino-N-phenethylpiperidine (4-ANPP), an immediate precursor for fentanyl, was selected as the substance of interest. A trial amount of 4-ANPP was 100 ng, and the amount of deposited dopant agent was 1 μg in each trial run. The trial runs were conducted for the following combinations: (a) TBSA only; (b) DEPA only; (c) 4-ANPP and TBSA as a dopant agent; (d) 4-ANPP and DEPA as a dopant agent; and (e) 4-ANPP only without any dopant agent. Plasmagrams were obtained for each separate trial run. FIG. 7 is a plot illustrating example ion mobility plasmagrams of samples including or excluding 4-ANPP in presence and absence of certain dopants, in accordance with one or more aspects of this disclosure.

As shown in curves (a) and (b), at room temperature, TBSA and DEPA form distinct ion peaks. When the substance of interest was present in the ionization source serving as a reaction chamber, adduct ions are formed with the dopant agents: [4-ANPP+TBSA+H]⁺shown in curve (c), and [4-ANPP+DEPA+H]⁺shown in curve (d). Curve (e) shows the plasmagram of pure 4-ANPP. 4-ANPP deposited at 100 ng without a dopant does not generate any noticeable ion peak, indicating the enhancement of sensitivity in accordance with one or more aspects of this disclosure.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

The present disclosure describes the following non-limiting enumerated aspects.
Aspect 1: A method for detecting an analyte in a sample, the method including: contacting the sample with at least one dopant, where the sample includes the analyte, where the at least one dopant includes a compound of Chemical Formula 1: R¹R²N—E(═O)Y′Y″ (Chemical Formula 1), where E is independently phosphorus (P), sulfur(S), or arsenic (As), where Y′ and Y″ are each independently a chemical species or a lone pair of electrons, where R¹ and R² are each independently selected from hydrogen, an alkyl group, an aryl group, a substituted alkyl group, or a substituted aryl group; forming an adduct ion from the analyte and the at least one dopant; performing spectrometric analysis of the adduct ion; and determining, based on the spectrometric analysis, the analyte.
Aspect 2: The method of aspect 1, where E is P, and where the at least one compound has Chemical Formula 2: R¹R²N—P(═O)(OR³)(OR⁴) (Chemical Formula 2), where R³ and R⁴ are each independently selected from hydrogen, an alkyl group, an aryl group, a substituted alkyl group, or a substituted aryl group.
Aspect 3: The method of aspects 1 or 2, where E is P, and where the at least one compound includes a phosphoramidic acid having Chemical Formula 3: H₂N—P(═O)(OH)₂ (Chemical Formula 3).
Aspect 4: The method of any of aspects 1 to 3, where E is P, and where the at least one compound includes a nucleotide phosphoramidate.
Aspect 5: The method of any of aspects 1 to 4, where E is S, where the at least one compound includes a sulfinamide, having Chemical Formula 4: R¹R²N—S(═O)—R³ (Chemical Formula 4).
Aspect 6: The method of any of aspects 1 to 5, where E is S, where the at least one compound includes a sulfonamide, having Chemical Formula 5: R¹R²N—SO₂—R³ (Chemical Formula 5).
Aspect 7: The method of any of aspects 1 to 6, where E is S, where the at least one compound includes a sulfamate, having Chemical Formula 6: R¹R²N—SO₂—O—R³ (Chemical Formula 6).
Aspect 8: The method of any of aspects 1 to 7, where E is S, and where the at least one compound has Chemical Formula 7: R¹R²N—S(═O)—O—R³ (Chemical Formula 7).
Aspect 9: The method of any of aspects 1 to 8, where the spectrometric analysis includes ion mobility spectrometry.
Aspect 10: The method of aspect 9, where the ion mobility spectrometry includes: introducing an adduct ion including an ionized product of the at least one dopant and the analyte in a drift chamber; generating, by an ion detector, a drift signal in response to detecting drifted ions at a predetermined position along the drift chamber; and by processing circuitry: determining, based on the drift signal, a sample drift time; and detecting the analyte in the sample by comparing the sample drift time with a target drift time.
Aspect 11: The method of aspect 10, further including, by the processing circuitry, generating an output indicative of the presence of the analyte, where the output includes at least one of a digital signal, an audio signal, or a display signal.
Aspect 12: The method of aspects 10 or 11, where the drift chamber is at room temperature.
Aspect 13: The method of aspects 10 or 11, where the drift chamber is at a temperature of 150° C. or less.
Aspect 14: The method of aspect 13, where the temperature is 80° C. or less.
Aspect 15: The method of any of aspects 1 to 14, further including ionizing the analyte and the at least one dopant to form the adduct ion.
Aspect 16: The method of any of aspects 1 to 15, further including fluidizing a dopant composition to generate the dopant.
Aspect 17: The method of any of aspects 1 to 16, where the spectrometric analysis includes mass spectrometry.
Aspect 18: The method of any of aspects 1 to 17, where the analyte includes a narcotic.
Aspect 19: The method of aspect 18, where the narcotic includes at least one of heroin, cocaine, tetrahydrocannabinol (THC), cannabidiol (CBD), xylazine, nitazenes, fentanyl, norfentanyl, carfentanil, acetyl fentanyl, acrylfentanyl, or a fentanyl analog.
Aspect 20: The method of any of aspects 1 to 19, where the analyte includes a chemical weapon substance.
Aspect 21: The method of aspect 20, where the chemical weapon substance includes at least one of tabun, sarin, soman, VX nerve agent, mustard gas, or a mustard gas derivative.
Aspect 22: An ion mobility spectrometry system including: a dopant chamber configured to fluidize a dopant composition to generate at least one dopant; a drift chamber configured to receive an adduct ion including an ionized product of the at least one dopant and an analyte, where a sample includes the analyte; an ion detector configured to generate a drift signal in response to detecting drifted ions at a predetermined position along the drift chamber; and processing circuitry configured to: determine, based on the drift signal, a sample drift time; and detect the analyte in the sample by comparing the sample drift time with a target drift time, where the at least one dopant includes a compound of Chemical Formula 1: R¹R²N—E(═O)Y′Y″ (Chemical Formula 1), where E is independently phosphorus (P), sulfur(S), or arsenic (As), where Y′ and Y″ are each independently a chemical species or a lone pair of electrons, where R¹ and R² are each independently selected from hydrogen, an alkyl group, an aryl group, a substituted alkyl group, or a substituted aryl group.
Aspect 23: The system of aspect 22, where the processing circuitry is further configured to generate an output indicative of the presence of the analyte, where the output includes at least one of a digital signal, an audio signal, or a display signal.
Aspect 24: The system of aspect 23, further including an ionization chamber configured to ionize the sample into an ionized sample and the at least one dopant into an ionized dopant, where the ionized product comprises the ionized sample and the ionized dopant.
Aspect 25: A portable spectrometry device including a handheld housing, the handheld housing including the ion mobility spectrometry system of any of aspects 22 to 24.
Aspect 26: A sampling swab pre-treated with at least one dopant agent, where the at least one dopant includes a compound of Chemical Formula 1: R¹R²N—E(═O)Y′Y″ (Chemical Formula 1), where E is independently phosphorus (P), sulfur(S), or arsenic (As), where Y′ and Y″ are each independently a chemical species or a lone pair of electrons, where R¹ and R² are each independently selected from hydrogen, an alkyl group, an aryl group, a substituted alkyl group, or a substituted aryl group.
Aspect 27: The sampling swab of aspect 26, where the at least one compound includes a compound of the method of any of aspects 2 to 8.
Aspect 28: The sampling swab of aspects 26 or 27, further including: a handle; and a swab portion configured to carry the sample.
Aspect 29: A method for detecting a chemical substance, the method comprising: collecting a sample containing a substance of interest; mixing the sample with at least one dopant agent in a reaction chamber, wherein the dopant agent comprises at least one element selected from phosphorus (P), sulfur(S), or arsenic (As), wherein the element is: (i) covalently bonded to oxygen via a double bond (E═O); and (ii) covalently bonded to nitrogen (E—NR¹R²), where E represents P, S, or As, and where R¹ and R² are each independently selected from hydrogen, an alkyl group, an aryl group, a substituted alkyl group, or a substituted aryl group; using an ionization source to generate an adduct ion formed from the substance of interest and the dopant, where the adduct contains the dopant and the substance of interest; performing spectrometric analysis of the adduct; and identifying the substance of interest based on the spectrometric data.
Aspect 30: The method of aspect 29, where the phosphorus-containing moiety is selected from: (a) phosphoramides, having the general formula R¹R²N—P(═O)(OR³)(OR⁴), where R³ and R⁴ are each independently selected from hydrogen, an alkyl group, an aryl group, a substituted alkyl group, or a substituted aryl group; (b) phosphoramidic acids, having the formula H₂N—P(═O)(OH)₂; or (c) phosphorylated biomolecules, including nucleotide phosphoramidates.
Aspect 31: The method of aspects 29 or 30, (a) sulfinamides, having the formula R¹R²N—S(═O)—R³; (b) sulfonamides, having the formula R¹R²N—O₂—R³; (c) sulfamates, having the formula R¹R²N—SO₂—O—R³; or (d) a compound having the formula R¹R²N—S(═O)—O—R³.
Aspect 32: The method of any of aspects 29 to 31, wherein the arsenic-containing moiety is selected from: (a) arsenamides, having the formula R¹—As(═O)—NR²R³; or (b) arsenic acid derivatives, including H₃AsO₄ and its esters.
Aspect 33: The method of any of aspects 29 to 32, where the spectrometric analysis involves ion mobility spectrometry.
Aspect 34: The method of aspect 33, wherein the ion mobility separation is performed at room temperature.
Aspect 35: The method of aspect 34, wherein the ion mobility separation is carried out at a temperature of up to 50° C., 60° C., 70° C., 80° C., 90° C., 100° C., 110° C., 120° C., 125° C., 130° C., 140° C., or 150° C.
Aspect 36: The method of aspect 35, wherein the temperature is up to 125° C.
Aspect 37: The method of aspect 35, wherein the temperature is up to 80° C.
Aspect 38: The method of any of aspects 29 to 37, where the spectrometric analysis involves mass spectrometry.
Aspect 39: The method of any of aspects 29 to 38, where the substance of interest includes an illicit drug substance.
Aspect 40: The method of aspect 39, where the illicit drug substance includes at least one of heroin, cocaine, tetrahydrocannabinol (THC), cannabidiol (CBD), xylazine, nitazenes, fentanyl, norfentanyl, carfentanil, acetyl fentanyl, acrylfentanyl, or all other fentanyl analogs.
Aspect 41: The method of any of aspects 29 to 40, where the substance of interest includes chemical weapon substance.
Aspect 42: The method of any of aspect 41, where the chemical weapon substance includes at least one of tabun, sarin, soman, VX nerve agent, mustard gas, or mustard gas derivatives.
Aspect 43: A system configured to perform the method as described in any of aspects 29 to 42.
Aspect 44: A sampling swab pre-treated with at least one dopant of the method of any of aspects 29 to 42.
Aspect 45: A dopant of the method of any of aspects 29 to 42.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

Based upon the above discussion and illustrations, it is recognized that various modifications and changes may be made to the disclosed technology in a manner that does not necessarily require strict adherence to the examples and applications illustrated and described herein. Such modifications do not depart from the true spirit and scope of various aspects of the disclosure, including aspects set forth in the claims.

What is claimed is:

1. A method for detecting an analyte in a sample, the method comprising:
contacting the sample with at least one dopant, wherein the sample comprises the analyte, wherein the at least one dopant comprises a compound of Chemical Formula 1: 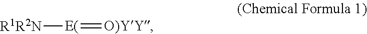
wherein E is independently phosphorus (P), sulfur(S), or arsenic (As), wherein Y′ and Y″ are each independently a chemical species or a lone pair of electrons; wherein R¹ and R² are each independently selected from hydrogen, an alkyl group, an aryl group, a substituted alkyl group, or a substituted aryl group;
forming an adduct ion from the analyte and the at least one dopant;
performing spectrometric analysis of the adduct ion; and
determining, based on the spectrometric analysis, the analyte.

2. The method of claim 1, wherein E is P, and wherein the at least one compound has Chemical Formula 2: 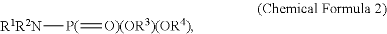
wherein R³ and R⁴ are each independently selected from hydrogen, an alkyl group, an aryl group, a substituted alkyl group, or a substituted aryl group.

3. The method of claim 1, wherein E is P, and wherein the at least one compound comprises a phosphoramidic acid having Chemical Formula 3:

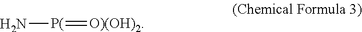

4. The method of claim 1, wherein E is P, and wherein the at least one compound comprises a nucleotide phosphoramidate.

5. The method of claim 1, wherein E is S, wherein the at least one compound comprises a sulfinamide, having Chemical Formula 4: 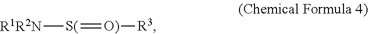
wherein R³ is independently selected from hydrogen, an alkyl group, an aryl group, a substituted alkyl group, or a substituted aryl group.

6. The method of claim 1, wherein E is S, wherein the at least one compound comprises a sulfonamide, having Chemical Formula 5: 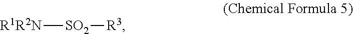
wherein R³ is independently selected from hydrogen, an alkyl group, an aryl group, a substituted alkyl group, or a substituted aryl group.

7. The method of claim 1, wherein E is S, wherein the at least one compound comprises a sulfamate, having Chemical Formula 6: 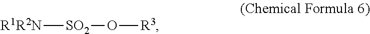
wherein R³ is independently selected from hydrogen, an alkyl group, an aryl group, a substituted alkyl group, or a substituted aryl group.

8. The method of claim 1, wherein E is S, and wherein the at least one compound has Chemical Formula 7: 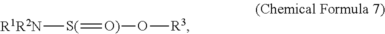
wherein R³ is independently selected from hydrogen, an alkyl group, an aryl group, a substituted alkyl group, or a substituted aryl group.

9. The method of claim 1, where the spectrometric analysis comprises ion mobility spectrometry.

10. The method of claim 9, wherein the ion mobility spectrometry comprises:
introducing an adduct ion comprising an ionized product of the at least one dopant and the analyte in a drift chamber;
generating, by an ion detector, a signal in response to detecting drifted ions ; and
by processing circuitry:
determining, based on the signal, a sample drift time; and
detecting the analyte in the sample by comparing the sample drift time with a target drift time.

11. The method of claim 10, further comprising, by the processing circuitry, generating an output indicative of the presence of the analyte, wherein the output comprises at least one of a digital signal, an audio signal, or a display signal.

12. The method of claim 10, wherein the drift chamber is at a temperature of 150° C. or less.

13. The method of claim 1, wherein the analyte comprises a narcotic.

14. The method of claim 13, wherein the narcotic comprises at least one of heroin, cocaine, tetrahydrocannabinol (THC), cannabidiol (CBD), xylazine, nitazenes, fentanyl, norfentanyl, carfentanil, acetyl fentanyl, acrylfentanyl, or a fentanyl analog.

15. The method of claim 1, wherein the analyte comprises a chemical weapon substance.

16. The method of claim 15, wherein the chemical weapon substance comprises at least one of tabun, sarin, soman, VX nerve agent, mustard gas, or a mustard gas derivative.

17. An ion mobility spectrometry system comprising:
a dopant chamber configured to fluidize a dopant composition to generate at least one dopant;
a drift chamber configured to receive an adduct ion comprising an ionized product of the at least one dopant and an analyte, wherein a sample comprises the analyte;
an ion detector configured to generate a signal in response to detecting drifted ions; and
processing circuitry configured to:
determine, based on the signal, a sample drift time; and
detect the analyte in the sample by comparing the sample drift time with a target drift time,
wherein the at least one dopant comprises a compound of Chemical Formula 1: 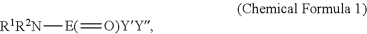
wherein E is independently phosphorus (P), sulfur(S), or arsenic (As), wherein Y′ and Y″ are each independently a chemical species or a lone pair of electrons; wherein R¹ and R² are each independently selected from hydrogen, an alkyl group, an aryl group, a substituted alkyl group, or a substituted aryl group.

18. The system of claim 17, wherein the processing circuitry is further configured to generate an output indicative of the presence of the analyte, wherein the output comprises at least one of a digital signal, an audio signal, or a display signal.

19. The system of claim 18, further comprising an ionization chamber configured to ionize the sample into an ionized sample and the at least one dopant into an ionized dopant, wherein the ionized product comprises the ionized sample and the ionized dopant.

20. A sampling swab pre-treated with at least one dopant agent, wherein the at least one dopant comprises a compound of Chemical Formula 1: 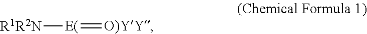
wherein E is independently phosphorus (P), sulfur(S), or arsenic (As), wherein Y′ and Y″ are each independently a chemical species or a lone pair of electrons; wherein R¹ and R² are each independently selected from hydrogen, an alkyl group, an aryl group, a substituted alkyl group, or a substituted aryl group.
